(19) United States
(12) Patent Application Publication
(10) Pub. No.: US 2010/0086590 A1
(43) Pub. Date: Apr. 8, 2010

(54) NOVEL STABLE PHARMACEUTICAL COMPOSITIONS OF CLOPIDOGREL BISULFATE AND PROCESS OF PREPARATION THEREOF

(75) Inventors: Deepak Anant Hegde, Thane (W) Maharashtra (IN); Santosh Sadashiv Chothe, Navi Mumbai (IN); Imran Shakur Ghogari, Hingoli (IN); Ashok Omray, Mumbai (IN)

Correspondence Address: SCHWEGMAN, LUNDBERG & WOESSNER, P.A., P.O. BOX 2938, MINNEAPOLIS, MN, 55402, US

(73) Assignee: USV Limited, Govandi Mumbai (IN)

(21) Appl. No.: 12/575,813

(22) Filed: Oct. 8, 2009

Related U.S. Application Data
(63) Continuation of application No. PCT/IN2008/000234, filed on Apr. 8, 2008.

(30) Foreign Application Priority Data
Apr. 9, 2007 (IN) 702/MUM/2007

(51) Int. Cl. A61K 9/20; A61K 31/4365; A61P 9/10

(52) U.S. Cl. 424/464; 514/301; 427/2.14; 424/400

(57) ABSTRACT

The present invention discloses novel stable oral pharmaceutical compositions comprising the active ingredient Clopidogrel bisulfate and hydrophilic polymers along with pharmaceutically acceptable excipients. Particularly, the said Clopidogrel bisulfate is crystalline Form 1 and the composition additionally comprises of one or more chelating agents and antioxidants. Further the invention relates to a novel process for preparation of stable pharmaceutical compositions wherein the Clopidogrel bisulfate Form I is coated with a hydrophilic polymer thereby providing an increased physical and chemical stability to the composition.

### CROSS-REFERENCES TO RELATED APPLICATIONS

This application is a continuation application of PCT/IN2008/000234, filed Apr. 8, 2008, and published on Oct. 16, 2008 as WO 2008/122994 A2, which claimed priority to Indian Application No. 702/MUM/2007, filed Apr. 9, 2007, which applications and publication are incorporated herein by reference and made a part thereof.

### TECHNICAL FIELD OF THE INVENTION

The present invention relates to novel stable oral pharmaceutical compositions comprising the active ingredient Clopidogrel bisulfate.

### BACKGROUND

Clopidogrel is a dextro-rotatory enantiomer of methyl alpha-5-(4,5,6,7-tetrahydro (3,2-c) thieno pyridyl) (2-chlorophenyl)-acetate. Clopidogrel is useful as a medicine for prophylaxis and treatment of thrombotic events such as coronary artery disease, peripheral vascular disease and cerebrovascular disease by acting as a platelet aggregation inhibitor.

U.S. Pat. No. 4,847,265 (EP281459) for the first time disclosed the dextro-rotatory enantiomer of methyl alpha-5-(4,5,6,7-tetrahydro (3,2-c) thieno pyridyl) (2-chlorophenyl)-acetate (Clopidogrel), its pharmaceutically acceptable salts, process of preparation thereof and compositions using the same. U.S. Pat. No. 4,847,265 discloses that the dextro-rotatory isomer possessed platelet aggregation inhibiting activity in comparison to the levo-rotatory isomer which is less active and less well tolerated.

However, no reference was made to the specific polymorphic forms of Clopidogrel bisulfate in U.S. Pat. No. 4,847,265. The synthetic process claimed in U.S. Pat. No. 4,847,265 led to the preparation of Clopidogrel bisulfate Form I which was later revealed in U.S. Pat. No. 6,429,210.

U.S. Pat. No. 6,429,210 discloses that Clopidogrel bisulfate can exist in different polymorphic crystalline forms which differ from each other in terms of stability, physical properties, spectral characteristics and the process for their preparation. The novel polymorph disclosed in U.S. Pat. No. '210 was named Crystalline Form II.

Tablets of Clopidogrel that are commercially available (Plavix®) contains the crystalline Form II of Clopidogrel bisulfate. Plavix® is administered as an oral tablet at a recommended dose of 75 mg once daily.

U.S. Pat. No. 6,914,141 discloses pharmaceutical tablets comprising Clopidogrel along with a lubricant selected from zinc stearate, stearic acid and sodium stearyl fumarate to prevent the sticking during compression.

US20050031691 discloses a composition with the advantages of easy administration combined with rapid dissolution of the active agent; achieved using nano particulate active agent and a gel forming substance.

Clopidogrel bisulfate Form I is unstable in the presence of moisture and elevated temperatures and gets converted spontaneously into Form II. This poses a major challenge in the development of stable pharmaceutical compositions using Clopidogrel bisulfate Form I.

While numerous pharmaceutical compositions containing Clopidogrel bisulfate for oral administration are available, there still exists a need for commercially acceptable compositions, which provides good physico-chemical stability and increased solubility.

### OBJECT OF THE INVENTION

The main object of the invention is to provide novel stable pharmaceutical compositions comprising the active ingredient Clopidogrel bisulfate Form I and hydrophilic polymer along with pharmaceutically acceptable excipients wherein Clopidogrel bisulfate Form I is coated or granulated with the hydrophilic polymer.

Another object of the invention is to provide novel stable pharmaceutical compositions comprising the active ingredient Clopidogrel bisulfate and hydrophilic polymers along with pharmaceutically acceptable excipients; wherein the composition additionally comprises of one or more chelating agents and antioxidants.

Another object of the invention is to provide pharmaceutical compositions with increased solubility and improved dissolution facilitated by using hydrophilic polymers.

Another object of the invention is to provide a process for preparation of stable pharmaceutical compositions wherein Clopidogrel bisulfate Form I is coated or granulated with the hydrophilic polymer thereby providing an increased physico-chemical stability to the composition.

### SUMMARY OF THE INVENTION

The present invention relates to novel stable oral pharmaceutical compositions comprising the active ingredient Clopidogrel bisulfate and hydrophilic polymers along with pharmaceutically acceptable excipients; wherein the composition additionally comprises of one or more chelating agents and antioxidants. Particularly, the said Clopidogrel bisulfate is crystalline Form 1.

The invention further relates to a novel process for preparation of stable pharmaceutical compositions wherein the Clopidogrel bisulfate Form I is coated with the hydrophilic polymer thereby providing an increased physical and chemical stability to the composition with improved dissolution.

The present invention discloses novel stable oral pharmaceutical compositions comprising the active ingredient Clopidogrel bisulfate and hydrophilic polymers along with pharmaceutically acceptable excipients and a novel process for preparation of said stable pharmaceutical compositions. Particularly, the said Clopidogrel bisulfate is crystalline Form 1.

According to one aspect, the invention provides novel stable oral pharmaceutical compositions comprising the active ingredient Clopidogrel bisulfate and hydrophilic polymers along with pharmaceutically acceptable excipients; wherein the composition additionally comprises of one or more chelating agents and antioxidants.

According to another aspect, the invention provides a novel process for preparation of stable pharmaceutical compositions wherein the Clopidogrel bisulfate Form I is coated with the hydrophilic polymer selected from a group consisting of one or more of hydroxypropyl methyl cellulose, hydroxypropyl cellulose and hydroxyethyl cellulose or mixtures thereof; thereby providing an increased physical and chemical stability to the composition. Pharmaceutical compositions prepared according to the said process provides improved solubility with improved dissolution.

### BRIEF DESCRIPTION OF THE DRAWINGS

FIG. 1 shows comparative dissolution profile of Plavix and tablets obtained according to Example 1.

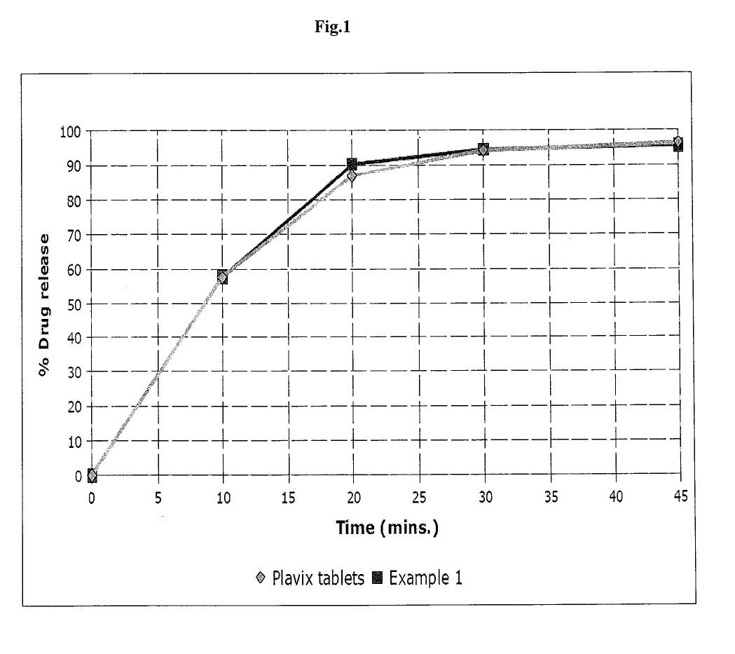

### DETAILED DESCRIPTION

The present invention describes a novel stable pharmaceutical composition comprising Clopidogrel bisulfate Form I and hydrophilic polymers along with pharmaceutically acceptable excipients wherein the Clopidogrel bisulfate Form I is coated with a hydrophilic polymer which provides a highly stable composition with improved dissolution. Said compositions further comprises one or more antioxidants and chelating agents. The invention further describes a process for the preparation of the said compositions.

Clopidogrel bisulfate Form I is unstable in the presence of moisture and elevated temperatures and gets converted spontaneously into Form II. This poses a major challenge in the development of stable pharmaceutical compositions using Clopidogrel bisulfate Form I. Further, Form I bulk solid is less compact and much more electrostatic than Form II and hence cannot be readily subjected to any treatment under the usual conditions of pharmaceutical technology. Moreover, Form I is practically insoluble in water and significant bioavailability can be a problem.

Despite the above mentioned drawbacks, the inventors of the present invention have successfully developed pharmaceutical compositions of Clopidogrel bisulfate Form 1 which provides both stability and improved solubility.

According to one embodiment, the present invention provides novel stable oral pharmaceutical compositions comprising the active ingredient Clopidogrel bisulfate and hydrophilic polymers along with pharmaceutically acceptable excipients; wherein the composition additionally comprises of one or more chelating agents and antioxidants.

According to another embodiment, the present invention provides novel stable pharmaceutical composition comprising Clopidogrel bisulfate Form I and hydrophilic polymers along with pharmaceutically acceptable excipients wherein the Clopidogrel bisulfate Form I is coated with a hydrophilic polymer. The resulting coated particles, storage granules or pellets exhibits improved stability at accelerated storage conditions.

In the practice of the present invention, the active ingredient Clopidogrel bisulfate Form I is used in the range of about 20.0% to about 70.0% by weight of the total composition. For example, the composition comprises Clopidogrel bisulfate Form I in the range of about 30.0% to about 50.0% by weight of the total composition. Clopidogrel bisulfate compositions of the present invention may be provided in dose strength of about 75 mg to about 300 mg and preferably in dose strength of 75 mg.

According to the present invention, the hydrophilic polymers are selected from cellulose derivative polymers. Cellulose derivative polymers that may be used are selected from a group consisting of one or more of hydroxypropyl methyl cellulose, hydroxypropyl cellulose and hydroxyethyl cellulose or mixtures thereof. Polymers having viscosity in the range of 3 to 100 cps are used. In one embodiment, the polymer is hydroxy propyl methyl cellulose with a viscosity in the range of 3 to 50 cps.

According to the invention, the hydrophilic polymer is present in the range of about 2.0% to about 50% by weight and preferably in the range of about 5.0% to 25.0% by weight of the total composition.

Hydrophilic polymers improves the solubility of the resultant formulation by reducing the contact angle and thus improves the dissolution of the formulation.

Additionally, the pharmaceutical compositions of the present invention comprises of one or more chelating agents and antioxidants. The presence of antioxidants and chelating agents helps to minimize the impurity formation caused by degradation of Clopidogrel bisulfate and thus improves the stability of the formulation.

In the practice of the present invention, water soluble and oil soluble antioxidants can be used. Water soluble antioxidants used as per the present invention are selected from a group consisting of sodium salts of bisulphite, sulphite, metabisulphite, thiosulphate, formaldehyde sulphoxylate, l and d ascorbic acid, cysteine, acetylcysteine, thioglycerol, thioglycollic acid,thiolactic acid, thiourea, dithithreitol, glutathione, or mixtures thereof. Oil soluble antioxidants are selected from a group consisting of propyl gallate, butylated hydroxy anisole, butylated hydroxy toluene, ascorbyl palmitate, nordihydroguaiaretic acid and alpha-tocopherol or mixtures thereof. The amount of antioxidant used is in the range of about 0.01% to about 1.00% by weight.

The chelating or sequestering agents are selected from a group consisting of edetic acids and its salts such as sodium salt of ethylene diamine tetra acetic acid, beta-hydroxyethylenediamine triacetic acid, diethylene triaminepentaacetic acid and nitrilotriacetate or mixtures thereof. The amount of chelating agent used is in the range of about 0.01% to about 1.00% by weight. Preferred chelating agent is sodium salt of ethylene diamine tetra acetic acid.

Compositions of the present invention may contain one or more pharmaceutically acceptable excipients selected from diluents, binders, lubricants, glidants, coating agents and the like.

Pharmaceutically acceptable carriers or diluents that are used for tabletting are selected from a group consisting of lactose monohydrate, lactose anhydrous, microcrystalline cellulose, mannitol and sugars or a mixture thereof. Diluents that are used in the formulation are anhydrous with below 3% moisture content which minimizes the chances of degradation. The pharmaceutical compositions of the present invention posses moisture content below 3%.

The amount of diluents used is in the range of about 20.0% to about 90.0% by weight. Microcrystalline cellulose can be used as a diluent as it provides good compressibility; for example, an anhydrous grade of microcrystalline cellulose can be used.

Lubricants that are used are selected from a group consisting of hydrogenated vegetable oil and siliconised talc, poloxomer 407 or a mixture thereof. Siliconised talc is mixture of Simethicone (3.0% to 10.0%) adsorbed on 90.0% of talc. The amount of lubricants used is in the range of about 1.0% to about 10.0% by weight.

Disintegrants that may be used include, but are not limited to crospovidone, croscarmellose sodium, sodium starch glycolate, sodium alginate and the like.

The polymer coated granules are further compressed with other pharmaceutically acceptable excipients and then film coated with a suitable coating agent. The amount of coating material used may be in the range from about 2.0% to about 5.0%.

Coating may be carried out using coating agents such as Opadry. Opadry contains hydroxypropyl methyl cellulose, plasticizers selected from triacetin, triethyl citrate, polyethylene glycol, opacifiers such as titanium dioxide. In one embodiment, the opadry is opadry pink which contains hydroxypropyl methyl cellulose, titanium dioxide, triacetin, iron oxide red, FD&C yellow/sunset yellow aluminum lake and iron oxide yellow. Solvents that may be used for coating include isopropyl alcohol and methylene dichloride.

Pharmaceutical compositions of the present invention are stable even at accelerated conditions of stability.

According to another embodiment, the invention provides a process for preparing Clopidogrel bisulfate compositions, the said process comprising the steps of:
(a) mixing clopidogrel bisulfate Form I with one or more chelating agents and antioxidants and pharmaceutically acceptable excipients;
(b) preparing a coating solution of hydrophilic polymer by dissolving the polymer in a mixture of isopropyl alcohol and methylene dichloride;
(c) coating or granulating the above mixture in step (a) with the coating solution of step (b) to form wet mass;
(d) drying the wet mass and further sizing the dried mass to form granules;
(e) blending and lubricating the sized granules to form mixture;
(f) compressing the lubricated mixture; and
(g) further coating the compressed tablets.

Compositions may be formulated by dry granulation, wet granulation or even direct compression. The pharmaceutical composition of the present invention can be formulated into a tablet.

In the practice of the present invention, the coating or granulation of Clopidogrel bisulfate may be carried out in equipments such as a fluid bed processor. The use of a fluid bed processor is advantageous as both granulations and drying can be performed in the same equipment. Sifting of the dried granules may be done using any sifter such as a vibro sifter. Compression is done using any conventional compression machine like rotary compression machine. Tablets may be compressed using suitable punches and dies to get tablets of required shape and size. The coating can be performed according to any of the conventional methods of coating using suitable coating agents and purified water or organic solvents.

The process of preparation as described herein is advantageous as it is industrially feasible and further the process of preparation results in decreased tendency of the material sticking to the surface of tooling during compression resulting in ease of manufacture.

Pharmaceutical compositions of the present invention are useful as a medicine for prophylaxis and treatment of thrombotic events such as coronary artery disease, peripheral vascular disease and cerebrovascular disease as it acts as a platelet aggregation inhibitor.

The present invention further provides the use of the pharmaceutical compositions in the prophylaxis and treatment of thrombotic events such as coronary artery disease, peripheral vascular disease and cerebrovascular disease by acting as a platelet aggregation inhibitor.

According to one embodiment, the present invention provides a method for treating a patient suffering from thrombotic events such as coronary artery disease, peripheral vascular disease or cerebrovascular disease comprising administering a therapeutically effective amount of Clopidogrel bisulfate composition prepared according to the present invention.

As used herein, the term “therapeutically effective amount” refers to an amount sufficient to cause an improvement in a clinically significant condition in the patient or even prevent a disease, disorder or condition in a patient.

As used herein, the term “excipients” refers to a pharmaceutically acceptable ingredient that is commonly used in the pharmaceutical technology for preparing, for example, granules or solid oral dosage formulations.

As used herein the term “tablet” is intended to encompass compressed pharmaceutical dosage forms of all shape and size, whether coated or uncoated.

The following examples are offered by way of illustration and not by way of limitation. The disclosures of all citations in the specification are expressly incorporated herein by reference.

### EXAMPLES

### Example 1

Clopidogrel bisulfate (Form I) (97.875 g), sodium metabisulphite (1.000 g) and disodium edetate (2.000 g) were mixed in a suitable equipment. This mix was granulated or coated with a coating solution prepared by dissolving hydroxypropyl methyl cellulose (10.000 g) in a mixture of isopropyl alcohol and methylene dichloride. The wet mass was then dried and sized. Sized granules were then mixed with microcrystalline cellulose (106.625 g) and crospovidone (15.000 g). The blend was further lubricated with siliconised talc (10.000 g) and hydrogenated vegetable oil (7.500 g). The said blend was compressed into tablets on rotary tablet press and the compressed tablets were coated with Opadry dispersion in water, hydro-alcoholic or organic solvents.

### Example 2

Clopidogrel bisulfate (97.875 g), Disodium EDTA (4.000 g) and Sodium metabisulfite (0.200 g) were mixed in a suitable equipment. Hydroxypropyl methyl cellulose (5.000 g) was dissolved in a mixture of isopropyl alcohol and methylene dichloride and was used for coating/granulation of the above dry mix. The wet mass was dried in fluid bed drier and sized to get the granules. Sized granules were mixed with microcrystalline cellulose (117.925 g), sodium starch glycolate (15.000 g) and lubricated using siliconised talc (10.000 g) as lubricant. The lubricated blend was compressed on tablet press to get the tablets. The tablets were coated using non aqueous dispersion of Opadry pink.

### Example 3

Clopidogrel bisulfate (97.875 g), microcrystalline Cellulose (176.825 g), Crospovidone (10.000 g) were mixed in a suitable equipment. Propyl gallate (0.100 g), butylated hydroxyl anisole (0.200 g) and hydroxypropyl cellulose (5.000 g) were mixed in a mixture of isopropyl alcohol and methylene dichloride and was used for granulation of above dry mix. The wet mass was dried in fluid bed drier and sized to get the granules. Sized granules were lubricated using Poloxamer 407 (10.000 g) as lubricant. The lubricated blend were compressed on tablet press to get the tablets. The tablets were coated using aqueous dispersion of Opadry pink.

### Example 4

Clopidogrel bisulfate (97.875 g), mannitol (181.825 g) and crospovidone (10.000 g) were mixed in suitable equipment. Sodium metabisulfite (0.300 g) and hydroxypropyl cellulose (5.000 g) were mixed in a mixture of isopropyl alcohol and methylene dichloride and was used for granulation of above dry mix. The wet mass was dried in fluid bed drier and sized to get the granules. Sized granules were lubricated using hydrogenated vegetable oil (5.000 g) as lubricant. The lubricated blend was compressed on tablet press to get the tablets. The tablets were coated using aqueous dispersion of Opadry pink.

The tablets prepared according to the Example No. 1 were analyzed for the impurities and the results obtained were compared with the Plavix tablets and is shown in Table 1. Dissolution was carried out in pH 2.0 acid buffer, 1000 ml and by using USP Type II method (paddle) at 50 rpm.

TABLE 1

| | Total | Dissolution (% release) | | | |
|---|---|---|---|---|---|
| | impurities | 10 mins | 20 mins | 30 mins | 45 mins |
| Plavix tablets | 0.880% | 57.5 | 86.9 | 94.1 | 96.5 |
| Example 1 | 0.516% | 57.6 | 90.1 | 94.5 | 95.4 |

The tablets prepared according to the Example No. 1 were subjected to accelerated stability testing at 40° C./75% Relative Humidity and the impurities in the respective tablets were analyzed and the results obtained are as shown in Table 2.

TABLE 2

| | Impurities | |
|---|---|---|
| Example 1 | Single impurity (unknown %) | Total impurity (%) |
| Initial | 0.084 | 0.516 |
| 3M at 40° C./ | 0.092 | 0.768 |
| 75% RH | | |

While the present invention is described above in connection with preferred or illustrative embodiments, these embodiments are not intended to be exhaustive or limiting of the invention. Rather, the invention is intended to cover all alternatives, modifications and equivalents included within its spirit and scope, as defined by the appended claims.

We claim:

1. A stable pharmaceutical composition comprising clopidogrel bisulfate and hydrophilic polymer wherein the active ingredient, clopidogrel bisulfate is coated with hydrophilic polymer.

2. The composition as claimed in claim 1, wherein the hydrophilic polymer is a cellulose derivative selected from the group consisting of hydroxypropyl methyl cellulose, hydroxypropyl cellulose and hydroxyethyl cellulose.

3. The composition as claimed in claim 1, wherein the clopidogrel bisulfate is crystalline Form I.

4. The composition as claimed in claim 2, wherein the hydrophilic polymer has a viscosity in the range of 3-100 cps.

5. The composition as claimed in claim 1, wherein the clopidogrel bisulfate is present in an amount ranging from about 20.0% to about 70.0% by weight of the composition and the hydrophilic polymer is present in the range of about 2.0% to about 50.0% by weight of the composition.

6. The composition as claimed in claim 1, wherein the composition additionally comprises one or more chelating agent and/or one or more antioxidant.

7. The composition as claimed in claim 6, wherein the chelating agent is selected from the group consisting of sodium salt of ethylene diamine tetra acetic acid, beta-hydroxyethylenediamine triacetic acid, diethylene triaminepentaacetic acid, nitrilotriacetate or mixtures thereof.

8. The composition as claimed in claim 6, wherein the antioxidant is water soluble or oil soluble.

9. The composition as claimed in claim 8, wherein the water soluble antioxidant is selected from the group consisting of sodium salts of bisulphite, sulphite, metabisulphite or thiosulphate, formaldehyde sulphoxylate, l and d ascorbic acid, cysteine, acetylcysteine, thioglycerol, thioglycollic acid, thiolactic acid, thiourea, dithithreitol, glutathione or mixtures thereof and the oil soluble antioxidant is selected from the group consisting of propyl gallate, butylated hydroxy anisole, butylated hydroxy toluene, ascorbyl palmitate, nordihydroguaiaretic acid and alpha-tocopherol or a mixture thereof.

10. The composition as claimed in claim 6, wherein the chelating agent is present in an amount of about 0.01 to about 1.00% by weight of the composition and the antioxidant is present in an amount of about 0.01% to about 1.00% by weight of the composition.

11. The composition as claimed in claim 6, wherein the chelating agent is sodium salt of ethylene diamine tetra acetic acid and the antioxidant is sodium metabisulphite.

12. The composition as claimed in claim 1 further comprises pharmaceutically acceptable excipients selected from diluents, binders, disintegrants, lubricants and coating agents.

13. The composition as claimed in claim 6, wherein the composition comprises about 30.0% to about 50.0% by weight of clopidogrel bisulfate, about 5.0% to about 25.0% by weight of hydrophilic polymer, about 0.01% to about 1.0% by weight of antioxidant and about 0.01% to about 1.0% by weight of chelating agent along with pharmaceutically acceptable excipients.

14. The composition as claimed in claim 12, wherein the diluent is selected from a group consisting of lactose monohydrate, lactose anhydrous, microcrystalline cellulose, mannitol, sugars or mixtures thereof.

15. The composition as claimed in claim 12, wherein the diluent has a moisture content below 3%.

16. The compositions as claimed in claim 15, wherein the composition has a moisture content below 3%.

17. The composition as claimed in claim 1, wherein the composition is in the form of a tablet.

18. A process for preparing stable pharmaceutical composition comprising clopidogrel bisulfate Form I, said process comprising the steps of:
(a) providing clopidogrel bisulfate Form I;
(b) preparing a coating solution of the hydrophilic polymer which is a cellulose derivative selected from the group consisting of hydroxypropyl methyl cellulose, hydroxypropyl cellulose and hydroxyethyl cellulose;
(c) coating clopidogrel bisulfate Form I with coating solution of step (b) to form polymer coated granules; and
(d) compressing the polymer coated granules into tablets.

19. The process as claimed in claim 18, further comprising mixing said clopidogrel bisulfate Form I with one or more chelating agents and/or one or more antioxidants before coating with the solution of step (b).

20. The process as claimed in claim 18, further comprising mixing said clopidogrel bisulfate Form I with at least one diluent before coating with the solution of step (b).

21. The process as claimed in claim 20, wherein the diluent is selected from a group consisting of lactose monohydrate, lactose anhydrous, microcrystalline cellulose, mannitol, sugars or mixtures thereof.

22. The process as claimed in claim 21, wherein the diluent is microcrystalline cellulose.

23. The process as claimed in claim 21, wherein the diluent has a moisture content below 3%.

24. The process as claimed in claim 18, wherein the coating solution is prepared by dissolving the polymer in a mixture of isopropyl alcohol and methylene dichloride.

25. The process as claimed in claim 24, wherein the coating solution further comprises one or more antioxidants.

26. The process as claimed in claim 18, wherein the tablets may optionally be coated.

27. The process as claimed in claim 18, further comprising mixing said polymer coated granules with at least one lubricant selected from the group consisting of hydrogenated vegetable oil, siliconised talc, poloxomer 407 or mixtures thereof before compression.

28. A method for treating a patient suffering from thrombotic events such as coronary artery disease, peripheral vascular disease or cerebrovascular disease comprising administering to the patient a composition as claimed in claim 1.
